(19) United States
(12) Patent Application Publication
(10) Pub. No.: US 2017/0105667 A1
(43) Pub. Date: Apr. 20, 2017

(54) Stress and Heart Rate Trip Monitoring System and Method

(71) Applicants: Josh WEI, Wilmette, IL (US); Parin PATEL, Mountain View, CA (US); Ida WAHLQUIST, Mountain View, CA (US)

(72) Inventors: Josh WEI, Wilmette, IL (US); Parin PATEL, Mountain View, CA (US); Ida WAHLQUIST, Mountain View, CA (US)

(21) Appl. No.: 14/884,665

(22) Filed: Oct. 15, 2015

(51) Int. Cl. A61B 5/16; G06F 19/00; A61B 5/00; A61B 5/0205; A61B 5/11

(52) U.S. Cl. CPC A61B 5/165; A61B 5/02055; A61B 5/1112; A61B 5/743; G06F 19/3418; A61B 5/024

(57) ABSTRACT

A trip stress monitoring method and device comprise receiving a geo-location data point, receiving a physiological measurement of a user and associating the physiological measurement to the received geo-location data point, storing the received geo-location data point and associated physiological measurement, continuing receiving geo-location data points and associated physiological measurements, displaying a map superimposed with the stored geo-location data points graphically forming a travel route, displaying a graphical representation of stored physiological measurements having a plurality of segments each representing an average physiological measurement value, and correlating each segment in the graphical representation to points in the travel route shown on the map.

### FIELD

The present disclosure relates to a mobile application, method, and device, and in particular in the field of stress and heart rate trip monitoring.

### BACKGROUND

In one nationwide survey, Eight out of ten drivers ranked aggressive driving as a “serious” or “extremely serious” risk that jeopardizes their safety. Statistically, aggressive driving accounts for more than half of all traffic fatalities. As roads become more congested and more drivers using the roads for their daily commutes in some city centers, incidents of road rage are on the rise. Many drivers feel extreme stress when they are stuck in traffic, get cut off by other drivers, and encounter rude drivers.

Stress isn't always bad. Stress within your comfort zone can help us perform under pressure and motivate us to do our best. However, when stress becomes overwhelming, it can damage our health, our mood, our productivity, our relationships, and our quality of life. Under stress our body releases chemicals that can shut down our ability to think, feel and act and hamper our body's ability to repair itself. For some people, untreated chronic stress can result in serious health conditions including anxiety, insomnia, muscle pain, high blood pressure and a weakened immune system.

### BRIEF DESCRIPTION OF THE DRAWINGS

FIG. 1 is a simplified block diagram of an exemplary embodiment of a stress and heart rate trip monitoring system and method according to the present disclosure;

FIG. 2 is a simplified flowchart of an exemplary embodiment of a stress and heart rate trip monitoring system and method according to the present disclosure;

FIGS. 3-5 are exemplary screen shots of a stress and heart rate trip monitoring system and method according to the present disclosure;

FIG. 6 is another simplified flowchart of an exemplary embodiment of a stress and heart rate trip monitoring system and method according to the present disclosure;

FIGS. 7-13 are further exemplary screen shots of a stress and heart rate trip monitoring system and method according to the present disclosure;

FIG. 14 is another simplified flowchart of an exemplary embodiment of a stress and heart rate trip monitoring system and method according to the present disclosure; and

FIG. 15 is a simplified block diagram of an exemplary mobile computing device 14 according to the teachings of the present disclosure.

### DETAILED DESCRIPTION

FIG. 1 is a simplified block diagram of an exemplary embodiment of a stress and heart rate trip monitoring system and method 10 according to the present disclosure. A mobile application 12 is adapted for execution on one or more mobile computing devices 14, including a mobile telephone, a wearable device, a vehicle, and other suitable computing devices. The mobile application 12 and the mobile computing device 14 are further in wireless communications (e.g., Bluetooth or another suitable protocol) with a physiological parameter monitor 16 that may be worn by the user or in the user's vicinity. The physiological parameter monitor 16 is operable to measure at least one physiological parameter of the user, including, but not limited to, heart (pulse) rate, body temperature, respiratory rate, blood pressure, perspiration, and facial expression, etc. that may be analyzed for indications of mental stress, anger, or anguish. The physiological parameter monitor 16 may be worn by the user around the wrist, ankle, forehead, face, waist, chest, body, etc. or incorporated into a piece of clothing or headgear. The physiological parameter monitor 16 may also be located somewhere close to the user's vicinity. The physiological parameter monitor may include a camera that may capture facial expressions of the user and using analysis to determine the emotion and stress level of the user. For example, images of the user's facial expression may show a relaxed, happy (smiling or laughing), angry, upset, sad, and sleepy countenance that may be analyzed to yield a stress level of the user.

The mobile application 12 and the mobile computing device 14 may be in communication with remote servers and/or databases 18 via a telecommunication network and the Internet 20 to access data as well as store data. Further, the mobile application 12 further includes a GPS utility or function in communication with the Global Positioning System (GPS) satellite constellation 22 to determine the present geographical location of the mobile computing device 14. The mobile application 12 further includes a mapping function that enables the display of a map on the screen of the mobile computing device 14. Alternatively the mobile application 12 may include an API (application programming interface) that provides an interface to an existing mapping algorithm, such as Google Maps. The GPS utility and the mapping function may be incorporated in one application that reside and execute on the mobile computing device 14.

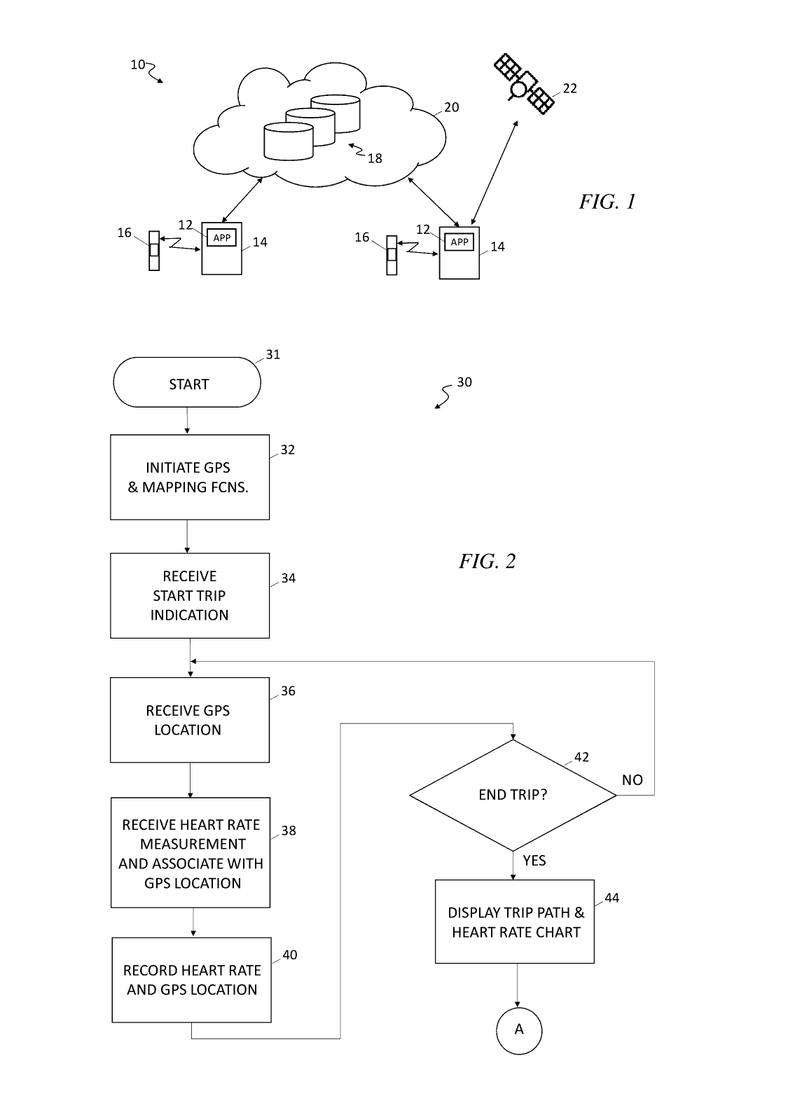

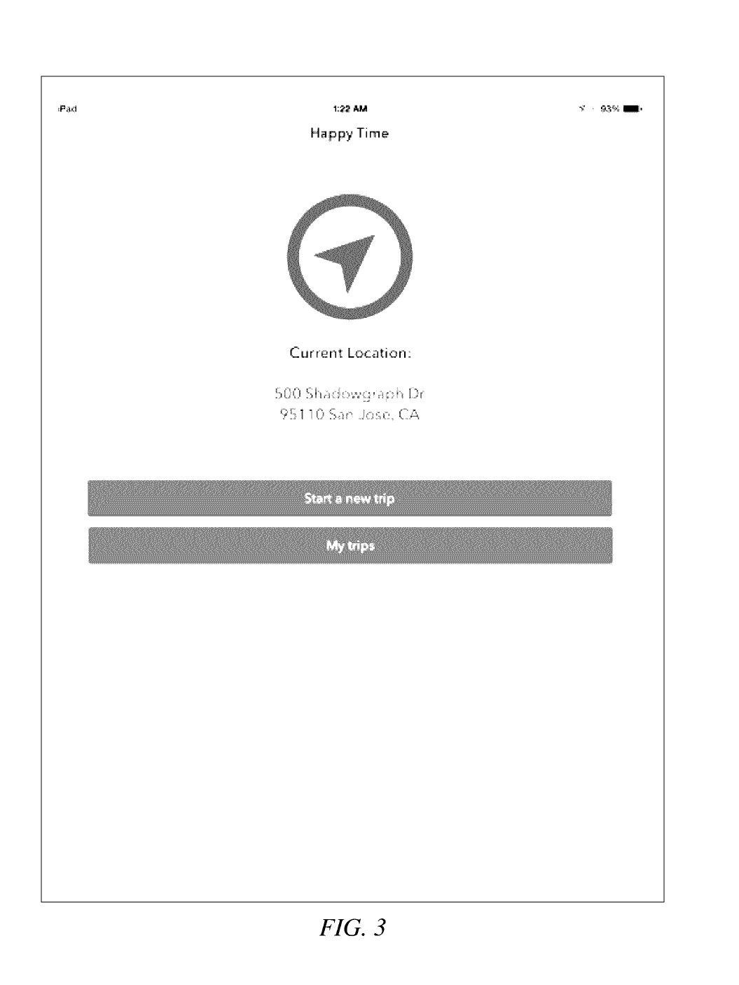

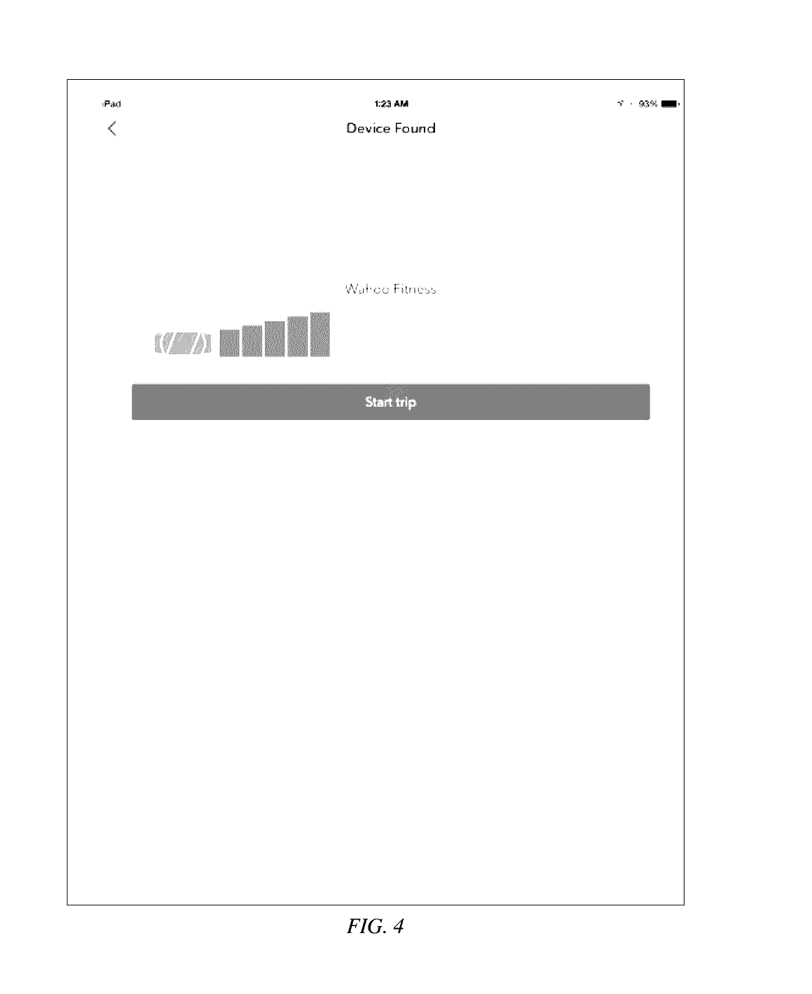

FIG. 2 is a simplified flowchart of an exemplary embodiment of a stress and heart rate trip monitoring system and method 30 according to the present disclosure. The method begins in block 31. In block 32, upon the start of the stress and heartrate trip monitoring application (upon execution of the application), the GPS and mapping functions are initiated. The screen may display the current location in the form of an address as shown in the exemplary screen shot in FIG. 3 and/or a map of the current location. In FIG. 4, the mobile computing device 14 is connected or in communication with the physiological parameter monitor 16. As shown in FIG. 4, the screen may display a graphical representation of the strength of the Bluetooth connection between the two devices. In block 34, a start trip input is received from the user. Alternatively, the method 30 may automatically begin the process. For example, the method 30 may start if it detects that the user is embarking on a familiar route, or simply that the user opened and began execution of the software application. The user may walk, jog, run, drive, or otherwise travel along a route (path, walkway, road, etc.). The mobile application 12 begins to receive and record the physiological parameter (heart rate) measurement and the GPS location data, as shown in blocks 36-40. The heart rate measurements are associated with the GPS locations so that the user's heart rate or physiological parameter value is known at every point of the trip. The physiological parameter and associated GPS location data are continually received and stored in memory until an indication of the end of the trip is received from the user, as shown in block 42. The data stored in memory may be continually uploaded to a remote server as they become available. Alternatively, the mobile application may operate without Internet connection and upload the user data when Internet communication becomes enabled or available.

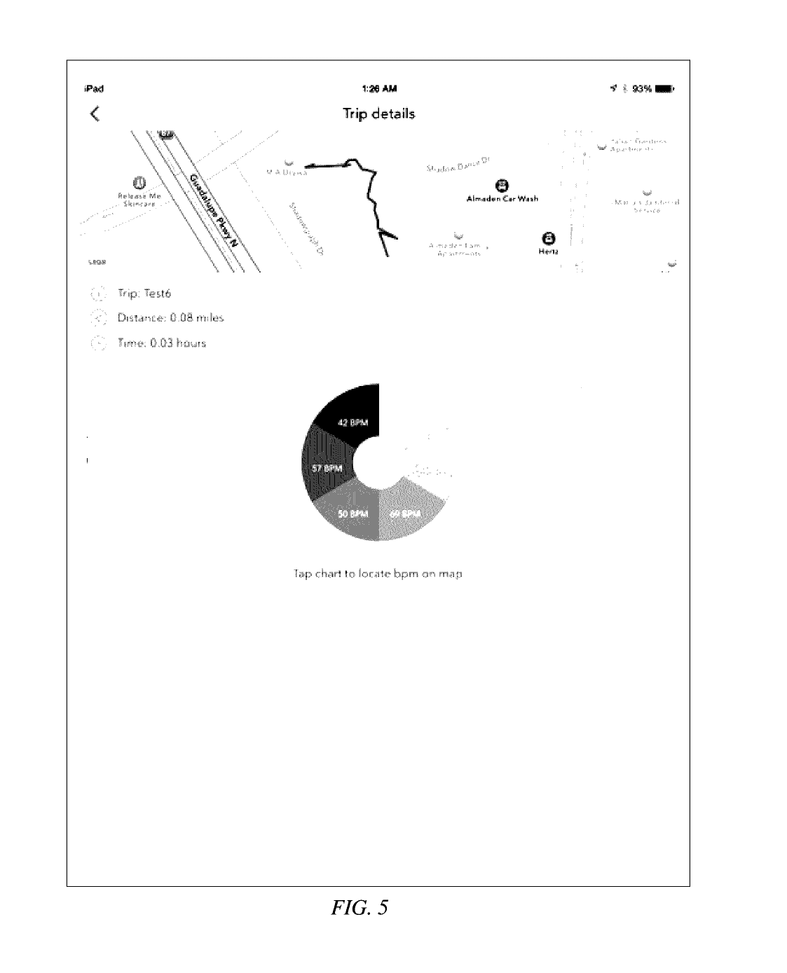

FIG. 5 is an exemplary screen shot of a stress and heart rate trip monitoring system and method according to the present disclosure. FIG. 5 shows an exemplary screen shot that may be displayed at the completion of the trip upon the user's stop input or automatic stop detection. Alternatively, the method 30 may automatically determine or detect a trip stopping point. A map is displayed with the route traveled by the user superimposed on the map. In addition, a graphical representation, such as in the form of a pie chart, shows representative average heart rate measurements (in beats per minute or BPM) recorded during the trip. The user may set a display preference for average heart rate, median heart rate, discrete heart rate values, etc. In the example shown in FIG. 5, the user had average measured heart rates of 42, 57, 50, 69, 40, and 89 BPM during this trip. The chart may additionally graphically represent proportionately the amount of time the user had spent with a certain heart rate. For example, the user may spend most of the time during the trip with a heart rate at 50 BPM. As a result, the pie chart may show 50 BPM occupying the largest percentage of the chart. Although the foregoing description focuses on displaying the heart rate, the user may selective change the display to show other physiological parameter measurements.

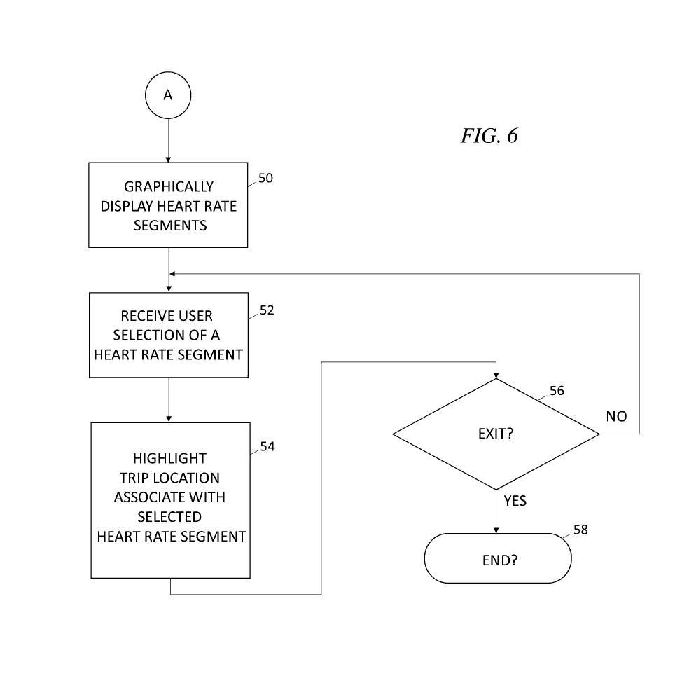

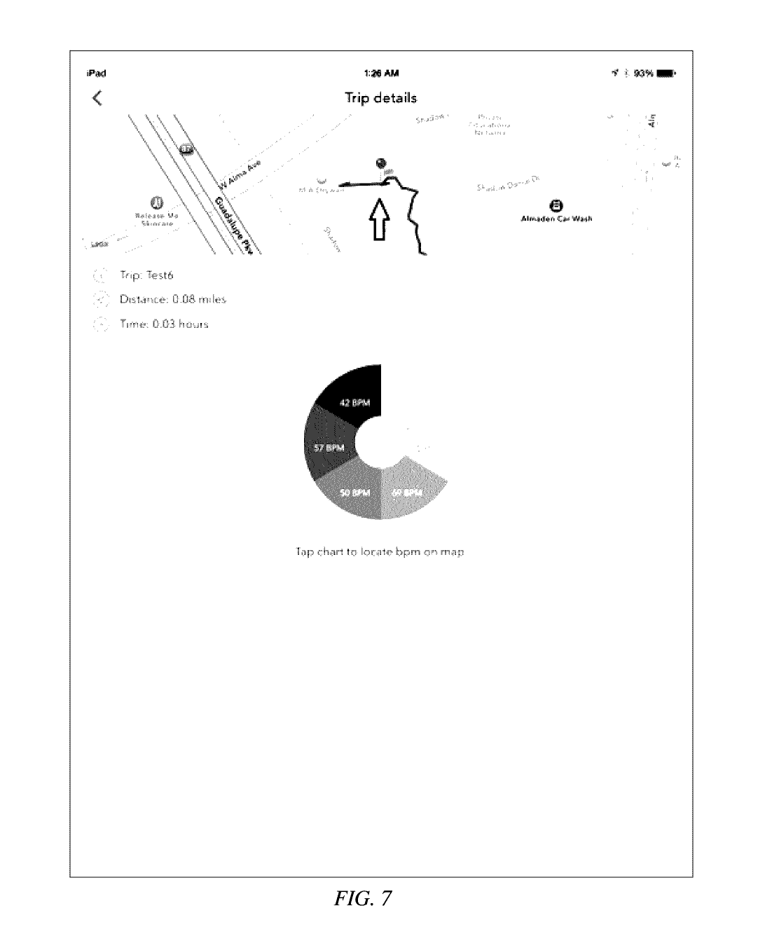

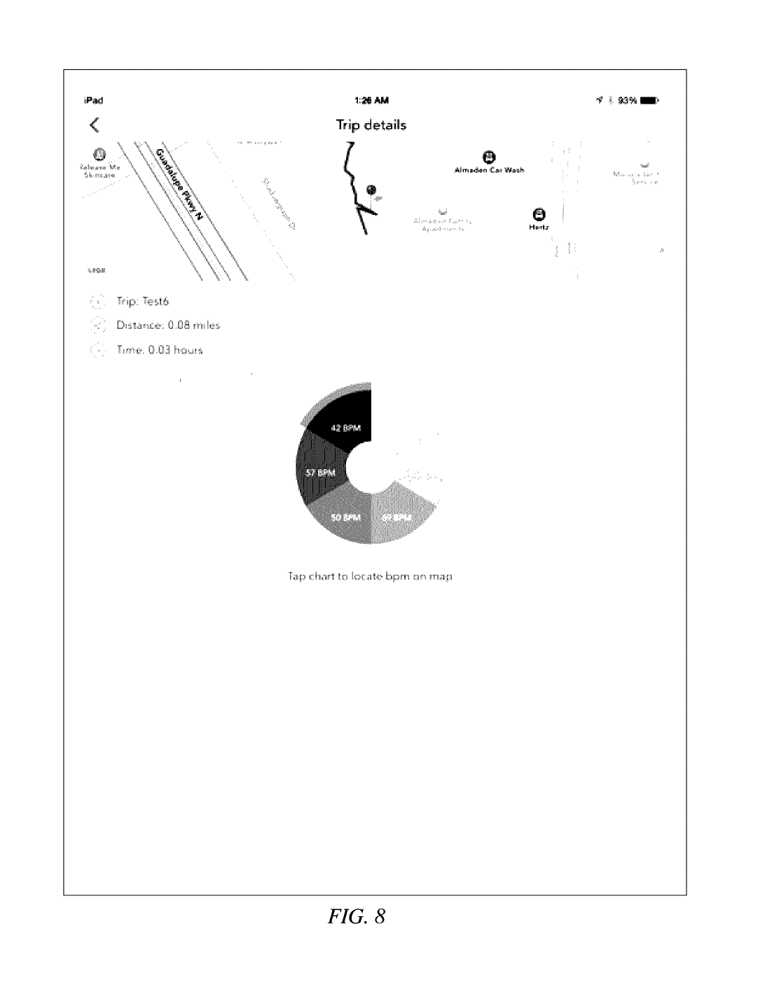

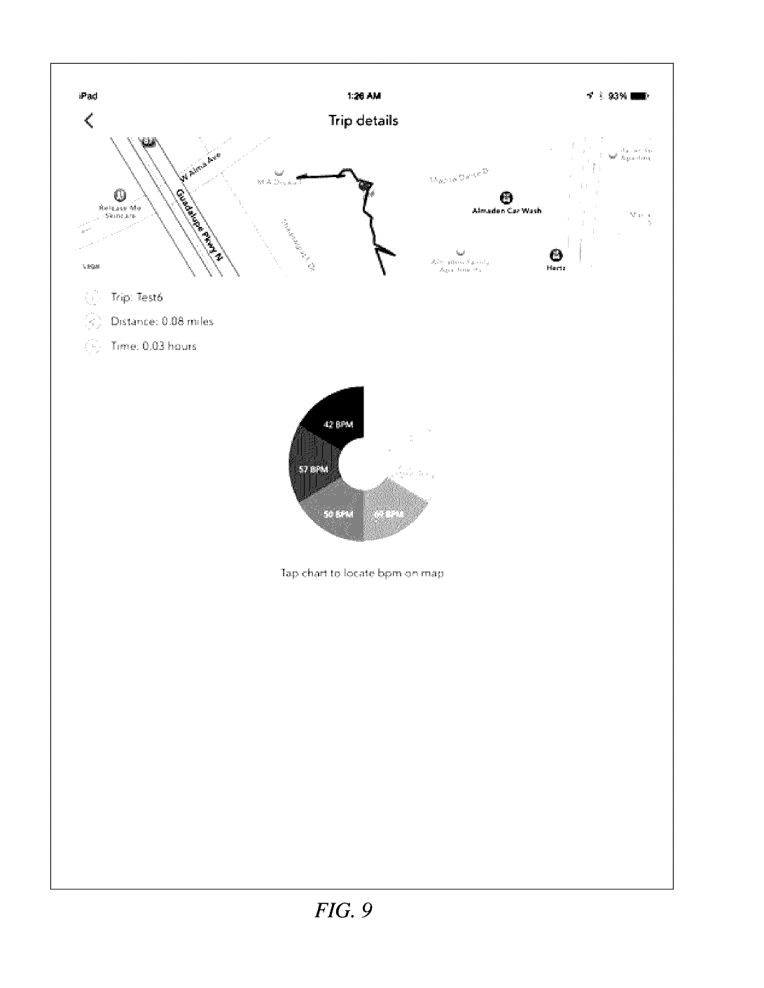

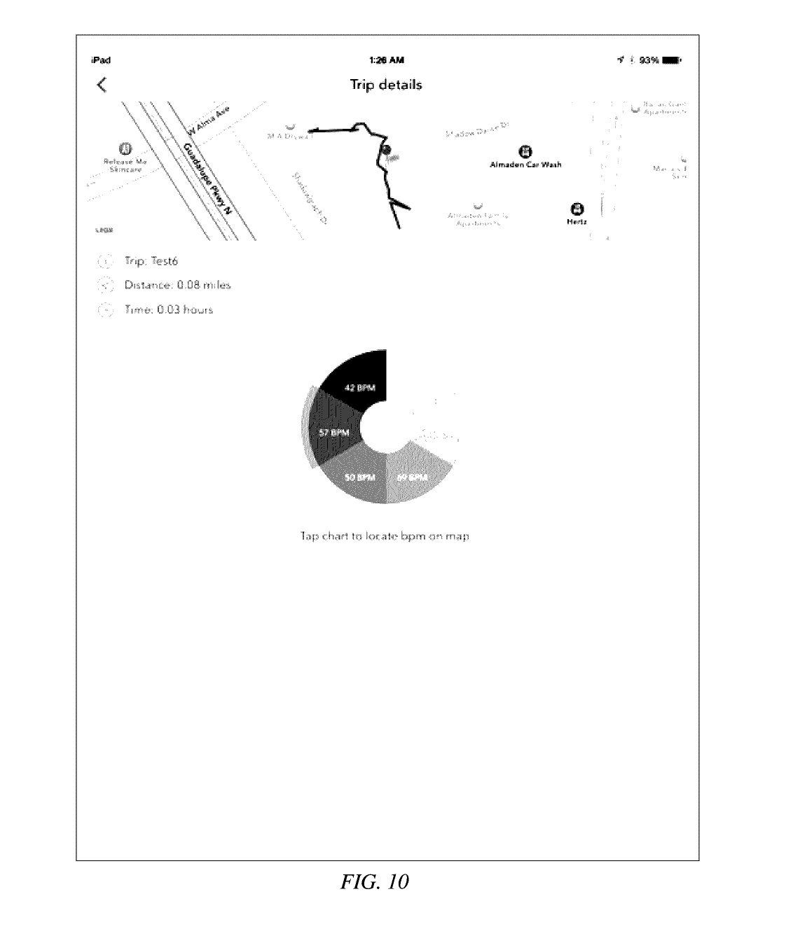

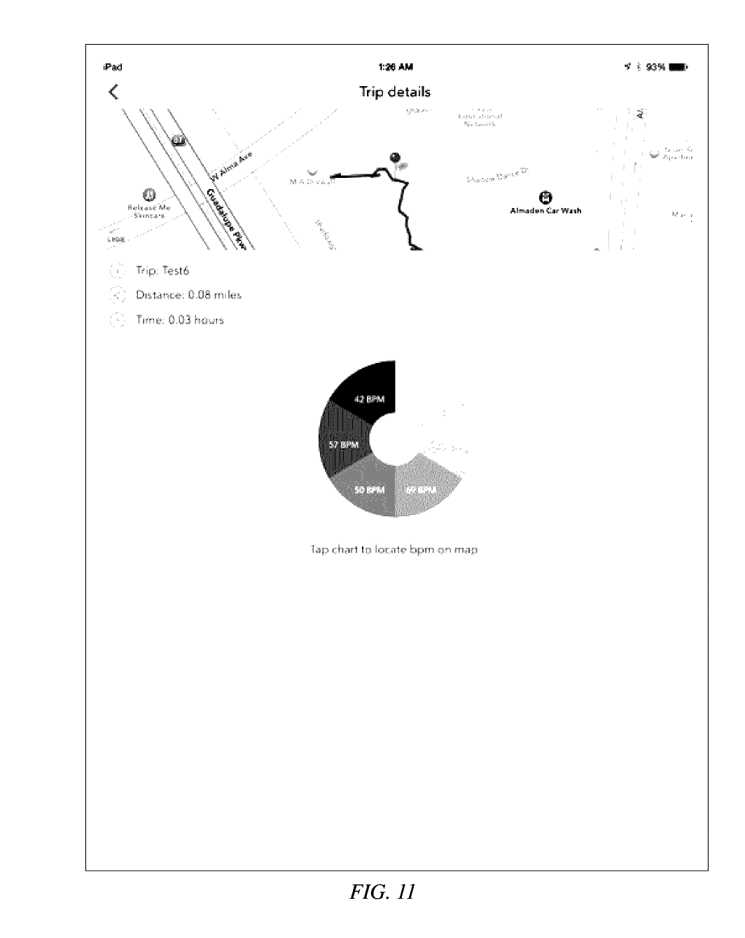

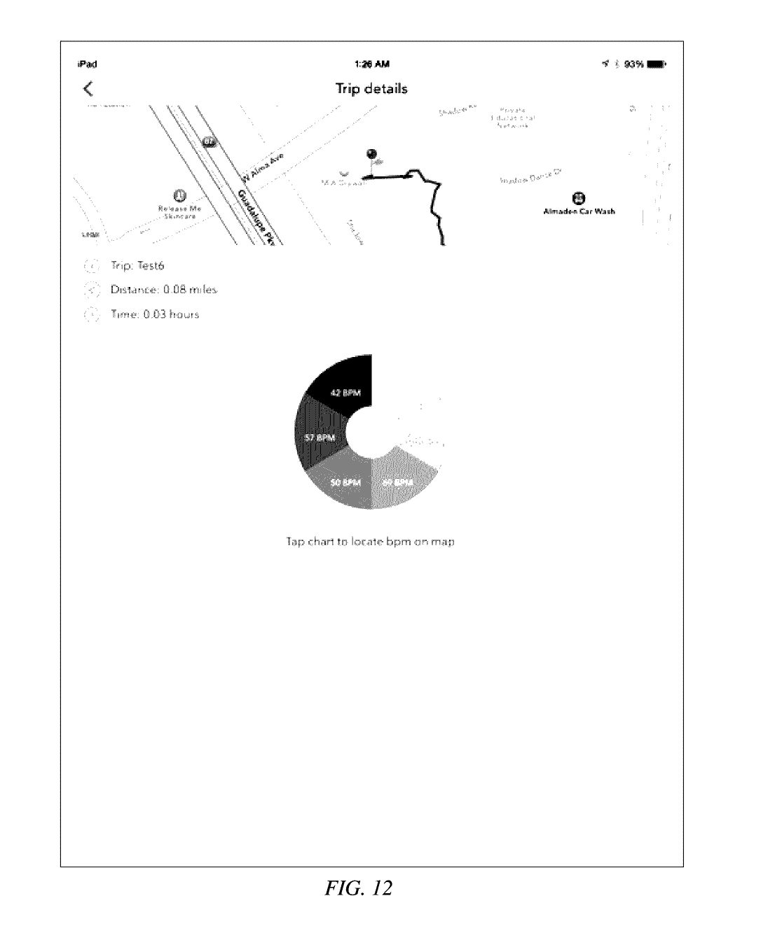

FIG. 6 continues from FIG. 2 and is another simplified flowchart of an exemplary embodiment of a stress and heart rate trip monitoring system and method according to the present disclosure. In block 50, the graphical representation or pie chart is shown with heart rate segments: 42, 57, 50, 69, 40, and 89 BPM. The mobile application 12 receives a user selection (e.g., click, touch, swipe, or voice input) of any displayed heart rate segment in the pie chart, as shown in block 52. In response, the mobile application 12 pinpoints a trip location that is associated with the selected heart rate. FIGS. 7-12 provide exemplary screen shots where different heart rate segments are selected and shown with the corresponding trip location.

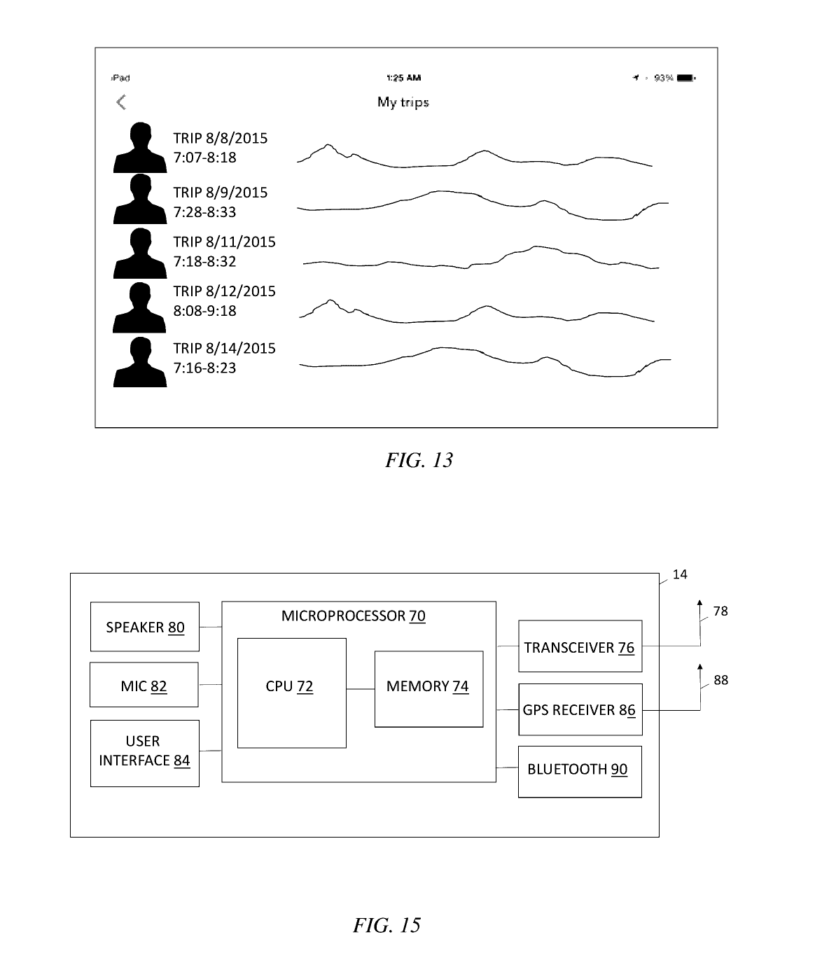

FIG. 13 is another exemplary screen shot of a stress and heart rate trip monitoring system and method according to the present disclosure. The screen display in FIG. 13 provides a data summary of past trips for this particular user. The list of trips is displayed with unique identifiers for the trips, dates, and start and end times. Additionally, this data summary may show the lowest and highest heart rates recorded during the trips, a graphical representation of heart rate changes, and/or a graphical representation of the terrain elevational profiles for the trips. The user may select a trip from the list, and the corresponding route map and graphical representation are displayed for review. Past trip data may be stored on the mobile computing device 14 or in a remote database 18 and accessible with user authentication.

By studying the trip data, the user is able to determine at which points during the route he/she is experiencing the highest level of mental and/or physical stress. The high heart rate may be due to the difficulty of the terrain traveled, the elevation changes in the route, or in the case of car travel, where another driver may have veered into the lane and nearly cause a collision or encountering a rude or discourteous driver. In the case of mental stress during the daily commute, for example, the user can pinpoint specific routes, intersections, or locations that cause high anxiety and stress, and can avoid them in future travels. For example, the user may recognize that his/her heart rate nearly always becomes elevated at a specific intersection at a certain time of the day. This intersection may be particularly congested due to pedestrian traffic or a bus stop where many buses pick up and drop off passengers, for example. As a result, the user can avoid this intersection in the future or employ de-stressing techniques (e.g., play soothing music) to dial down the stress level experienced during his/her commute. It should be noted that although the focus of the description herein is on heart rate measuring and monitoring, other physiological parameters may be measured and associated with the trip locations.

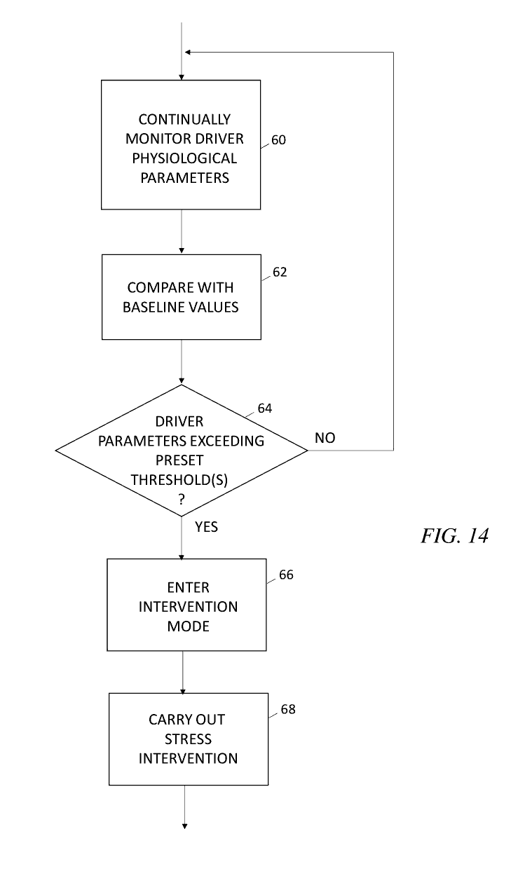

FIG. 14 is yet another simplified flowchart of an exemplary embodiment of a stress and heart rate trip monitoring system and method employing automatic intervention techniques according to the present disclosure. The system and method are capable of continually monitoring the user's physiological parameters and maintaining a set of baseline measurements, as shown in block 60. As described above, the physiological parameter monitor 16 is operable to measure at least one physiological parameter of the user, including, but not limited to, heart (pulse) rate, respiratory rate, body temperature, blood pressure, perspiration, etc. that may be analyzed for indications of mental stress, anger, or anguish. In block 62, the one or more measured values are compared with baseline measurements stored in memory. In block 64, if at least one of the current parameter values exceed or deviate from a preset threshold in comparison to the baseline measurement, e.g., the heart rate is more than 10% faster than the baseline heart rate measurement or the respiratory rate is more than 35 times per minute, then the system has identified physiological deviations and determined that the driver is experiencing a level of stress that warrants intervention. The system enters an intervention mode, as shown in block 66. In block 68, one or more intervention techniques are deployed. The user may have previously indicated or selected intervention preferences. For example, the user may prefer audio forms of intervention such as favorite song, audio track of a favorite comedian, recordings of loved ones (e.g., child saying “I love you, Mommy,” child's laughter), positive life affirming messages, etc. Another form of intervention may be visual, such as displaying still or moving images of breathtaking scenery, famous sites, family members, etc. Yet other forms of intervention may include regulating the interior temperature setting, adjusting/changing the driver's seat setting, initiate the massage/vibration functions of the driver's seat, etc. The intervention deployment may continue until the user's physiological parameters have returned to normal.

FIG. 15 is a simplified block diagram of an exemplary mobile computing device 14 according to the teachings of the present disclosure. The mobile computing device 14 includes a microprocessor 70 having a central processing unit 72 and memory 74. The microprocessor 70 is coupled to a transceiver 76 with an antenna 78 for wireless communication of data. The microprocessor 70 is further coupled to a speaker 80, a microphone 82, and a user interface 84 (e.g., touch screen, keypad, display screen). The microprocessor 70 is further coupled to a GPS receiver 86 and its antenna 88. A Bluetooth communication component 90 is further included in the mobile computing device 14. It should be noted that the mobile computing device 14 and the physiological parameter monitoring device 16 may be incorporated into one integrated device within a single housing.

The features of the present invention which are believed to be novel are set forth below with particularity in the appended claims. However, modifications, variations, and changes to the exemplary embodiments described above will be apparent to those skilled in the art, and the system and method described herein thus encompasses such modifications, variations, and changes and are not limited to the specific embodiments described herein.

What is claimed is:

1. A trip stress monitoring method, comprising:
determining a trip start point;
receiving a geo-location data point;
receiving a physiological measurement of a user and associating the physiological measurement to the received geo-location data point;
storing the received geo-location data point and associated physiological measurement;
continuing receiving geo-location data points and associated physiological measurements;
determining a trip end point;
displaying a map superimposed with the stored geo-location data points graphically forming a travel route;
displaying a graphical representation of stored physiological measurements having a plurality of segments each representing an average physiological measurement value; and
correlating each segment in the graphical representation to points in the travel route shown on the map.

2. The trip stress monitoring method of claim 1, wherein receiving a physiological measurement comprises receiving at least one of a heart rate, breathing rate, body temperature, blood pressure, perspiration, and facial image.

3. The trip stress monitoring method of claim 1, wherein displaying a graphical representation of stored physiological measurements comprises displaying a pie chart having a plurality of segments.

4. The trip stress monitoring method of claim 1, wherein correlating each segment in the graphical representation comprises:
receiving a user selection of a segment in the graphical representation; and
graphically indicating a point on the travel route a geo-location data point associated with the selected segment.

5. The trip stress monitoring method of claim 1, further comprising displaying a summary of past travel routes.

6. The trip stress monitoring method of claim 1, further comprising displaying geo-location data points and physiological measurements of a past travel route.

7. The trip stress monitoring method of claim 1, further comprising:
maintaining a set of baseline physiological measurements for the user;
receiving a set of current physiological measurements of the user;
comparing the set of current physiological measurements with the set of baseline physiological measurements; and
deploying an intervention in response to at least one of the current physiological measurements exceeding a preset threshold.

8. The trip stress monitoring method of claim 1, wherein deploying an intervention comprises deploying at least one of audio intervention, visual intervention, changing temperature setting, and changing driver's seat setting.

9. A non-transitory computer-readable medium having encoded thereon a trip stress monitoring method adapted to:
receive a start trip user input;
receive a geo-location data point;
receive a physiological measurement of a user and associating the physiological measurement to the received geo-location data point;
store the received geo-location data point and associated physiological measurement;
continue receiving geo-location data points and associated physiological measurements;
receive an end trip user input;
display a map superimposed with the stored geo-location data points graphically forming a travel route;
display a graphical representation of stored physiological measurements having a plurality of segments each representing an average physiological measurement value; and
correlate each segment in the graphical representation to points in the travel route shown on the map.

10. The non-transitory computer-readable medium of claim 9, wherein the method is adapted to receive at least one of a heart rate, body temperature, blood pressure, perspiration, and facial image.

11. The non-transitory computer-readable medium of claim 9, wherein the method is adapted to display a pie chart having a plurality of segments.

12. The non-transitory computer-readable medium of claim 9, wherein the method is further adapted to:
receive a user selection of a segment in the graphical representation; and
graphically indicate a point on the travel route a geo-location data point associated with the selected segment.

13. The non-transitory computer-readable medium of claim 9, wherein the method is further adapted to display a summary of past travel routes.

14. The non-transitory computer-readable medium of claim 9, wherein the method is further adapted to display geo-location data points and physiological measurements of a past travel route.

15. The non-transitory computer-readable medium of claim 9, wherein the method further comprises:
maintaining a set of baseline physiological measurements for the user;
receiving a set of current physiological measurements of the user;
comparing the set of current physiological measurements with the set of baseline physiological measurements; and
deploying an intervention in response to at least one of the current physiological measurements exceeding a preset threshold.

16. A trip stress monitoring device, comprising:
a user interface adapted to receive a start trip user input;
a GPS receiver adapted to receive a geo-location data point;
a wireless communication component adapted to receive a physiological measurement of a user;
a microprocessor adapted to receive the geo-location data point and the physiological measurement and associate the physiological measurement to the received geo-location data point;
a memory storing the received geo-location data point and associated physiological measurement;
the user interface adapted to receive an end trip user input;
a display screen adapted to display a map superimposed with the stored geo-location data points graphically forming a travel route;
the display screen adapted to display a graphical representation of stored physiological measurements having a plurality of segments each representing an average physiological measurement value; and
the display screen adapted to display a correlation between each segment in the graphical representation to points in the travel route shown on the map.

17. The trip stress monitoring device of claim 16, wherein the wireless communication component is adapted to receive at least one of a heart rate, body temperature, blood pressure, perspiration, and facial image.

18. The trip stress monitoring device of claim 16, wherein the display is adapted to display a pie chart having a plurality of segments.

19. The trip stress monitoring device of claim 16, further comprising:
the user interface adapted to receive a user selection of a segment in the graphical representation; and
the display screen adapted to visually indicate a point on the travel route a geo-location data point associated with the selected segment.

20. The trip stress monitoring device of claim 16, wherein the display screen is adapted to display a summary of past travel routes.

21. The trip stress monitoring device of claim 16, wherein the display screen is adapted to display geo-location data points and physiological measurements of a past travel route.

22. The trip stress monitoring device of claim 16, wherein the memory is adapted to maintain a set of baseline physiological measurements for the user, and the microprocessor is adapted to receive a set of current physiological measurements of the user, compare the set of current physiological measurements with the set of baseline physiological measurements, and deploy a user-selected intervention in response to at least one of the current physiological measurements exceeding a preset threshold.
